(19) United States
(12) Patent Application Publication
(10) Pub. No.: US 2023/0044611 A1
(43) Pub. Date: Feb. 9, 2023

(54) Reusable Breast Implant Delivery Apparatus with External Fluid Pressure Delivery System

(71) Applicant: SMART FUNNEL LLC, Houston, TX (US)

(72) Inventors: NANDHIKA WIJAY, Houston, TX (US); Arnab Roy, Houston, TX (US)

(73) Assignee: SMART FUNNEL LLC, Houston, TX (US)

(21) Appl. No.: 17/396,118

(22) Filed: Aug. 6, 2021

(51) Int. Cl. A61F 2/00; A61F 2/12

(52) U.S. Cl. CPC A61F 2/0095; A61F 2/12

(57) ABSTRACT

An implant delivery device suitable for delivery of breast implants features a reusable design that can be autoclaved to sterilize between uses. External fluid pressure is selectively delivered to a housing with the implant inside and can be regulated by the surgeon. The application of fluid pressure can be paused with the already applied pressure retained in the housing giving mechanical advantages for easier force application by the surgeon for enhanced control of the position of the implant within the housing and as it exits the outlet of the housing. The housing can be transparent to aid in observing the implant position and orientation as it moves through the housing. The outlet tip can be slant cut to allow for a fixed opening of the incision before delivering the implant to the surgical site. The housing cover is configured for rapid attachment with a seal that is amenable for autoclaving.

### FIELD OF THE INVENTION

The field of the invention is reusable implant delivery devices and more particularly devices for delivery of breast implants while using a pressurized fluid control system to receive, regulate or retain fluid pressure within the device providing mechanical advantages during breast implant movement through the device and into a surgical site.

### BACKGROUND OF THE INVENTION

In a breast implant procedure, the implant has been placed in a plastic device with flexible walls and is manipulated out of the device with a surgeon's hand toward an outlet for placing in the surgical site. These devices are designed for single use and as a result present a significant cost in the breast implant surgical procedure. These devices depend on a hand force from the surgeon in multiple localized regions on the exterior walls to transfer the force onto the implant. This applied force must be applied in a steady continuous manner to control the movement of the breast implant toward the device outlet. A release of the squeezing force as the implant is advancing to or through the outlet can result in the breast implant reversing motion direction and reorienting because the device housing is deliberately flexible under elastic deformation to allow a hand force to advance the breast implant to the surgical site. Due to the flexibility of such devices an implant can stretch the device causing trauma and ripping of the skin incision.

Some examples of such designs are the following patents and applications assigned to Keller Medical Inc: U.S. Pat. No. 9,168,126; US2015 0032208 and US2012 0143332. Indeed, US2015 0032208 teaches that the material for the housing is a heat shrink material that will collapse if there is an attempt to autoclave it, indicating the thinking of some skilled in the art that using the material for the housing responsive to hand squeezing to advance the breast implant makes it impossible to produce a reusable design that has to be autoclaved to prevent infection.

US2019 0365527 describes a device that uses the surgeon's hand to push a plunger within a housing that advances the breast implant through the housing outlet. The focus of this device is the shape of the housing conforming to the implant shape to maintain orientation of the implant relative to the surgical site as the breast implant exits the housing. An intermediate fluid between the hand operated piston and the implant is described to enable the piston stroke limit to advance the breast implant out of the housing. In this design, any force relaxation by the surgeon on the piston can result in reverse movement of the implant. This reversing of movement can reorient the breast implant within the housing or could cause folds or leak paths for the fluid that is between the piston and the breast implant.

Other related references comprise: U.S. Pat. No. 7,935,089B2; EP3087951A1; U.S. Pat. No. 9,610,152B1; U.S. Pat. No. 5,201,779A; WO2012177587A1; and U.S. Pat. No. 8,409,279B2.

What is needed and is addressed by the present invention is a design that allows reuse after autoclaving. Having such a feature can reduce the cost of surgical procedures involving breast implants. Since such procedures are not always covered by medical insurance, any device that can reduce the cost of the procedure overall can result in making the procedure more available to people who wish to undertake the surgery. Use of an external pressurized fluid supply to the housing with a feature to retain the delivered pressure takes the burden off the surgeon in several ways. The delivered pressure can be independent of the surgeon's hand thus allowing a steadier grip of the delivery housing during delivery of the breast implant. Additionally, the delivered pressurized fluid is retained in the housing thus preventing reversing movement of the breast implant. One way this is achieved is a check valve in the fluid delivery line that is connected to a quick disconnect cap such as a bayonet mount that involves a slight rotation to secure the cap to the housing under applied pressure. The retained input pressure up to three atmospheres but can be higher in some instances provides a mechanical advantage for the surgeon allowing for easier force application compared to hand squeeze or direct plunger device.

A seal between the cap and the housing can be incorporated into the design such as an o-ring in a groove or a flat gasket to seal the pressurized fluid that is applied. The outlet can be slant cut to split the incision in the breast prior to the breast implant advancing through the outlet port and into the now spread incision that leads to the pocket where the breast implant will be deposited before the incision is closed. The housing inlet is sized to readily accept implants of varying sizes with minimal, if any, manipulation in an atraumatic, direct transfer from implant package to housing technique. Multiple implants can also be placed inside such housing in a sequential delivery method. The housing can allow the implant to rest in its natural form prior to delivery. The outlet is smaller than the inlet in the housing to correlate with the incision length. The outlet can also be used with differing sizes of breast implants. Alternatively, the device can be used with a single correlating breast implant size. The fixed rigid outlet limits the possibility of stretching the skin incision and causing trauma.

While the device has its utility as a delivery device for breast implants, delivery of implants to other body locations is a contemplated use of the device as well. The housing can be made of metallic material or a transparent material that is amenable to be autoclaved. The see-through feature allows the surgeon to monitor the advancement of the breast implant within the housing as it approaches and exits the housing outlet. The interior wall of the housing is a low friction surface for easier implant delivery

These and other aspects of the present invention will be more readily understandable to those skilled in the art from a review of the description of the invention in greater detail below while recognizing that the full scope of the invention is to be determined from the appended claims.

### SUMMARY OF THE INVENTION

An implant delivery device suitable for delivery of breast implants features a reusable design that can be autoclaved to sterilize between uses. External fluid pressure is selectively delivered to a housing with the implant inside and can be regulated by the surgeon. The application of fluid pressure can be paused with the already applied pressure retained in the housing giving mechanical advantages for easier force application by the surgeon for enhanced control of the position of the implant within the housing and as it exits the outlet of the housing. The housing can be transparent to aid in observing the implant position and orientation as it moves through the housing. The outlet tip can be slant cut to allow for a fixed opening of the incision before delivering the implant to the surgical site. The housing cover is configured for rapid attachment with a seal between the cover and the housing that is amenable for autoclaving. Different sizes of outlets and inlets housing configurations allow for different size implants.

### BRIEF DESCRIPTION OF THE DRAWINGS

FIG. 1 is a section view of the apparatus with an implant within without pressure being applied;

FIG. 2 is the view of FIG. 1 with external fluid pressure applied;

FIG. 3 is an exploded view of the apparatus with an implant;

FIG. 3A is an alternative to FIG. 3 showing a handle on the cover and an alternate locking mechanism;

FIG. 4 is a perspective view of the apparatus of FIG. 1; and

FIG. 4A is an alternative to FIG. 4 showing an optional retractor holder

FIG. 5 is a top view of the apparatus of FIG. 4; and

FIG. 6 is a perspective view of an alternative housing shape to the version shown in FIG. 1.

### DETAILED DESCRIPTION OF THE PREFERRED EMBODIMENT

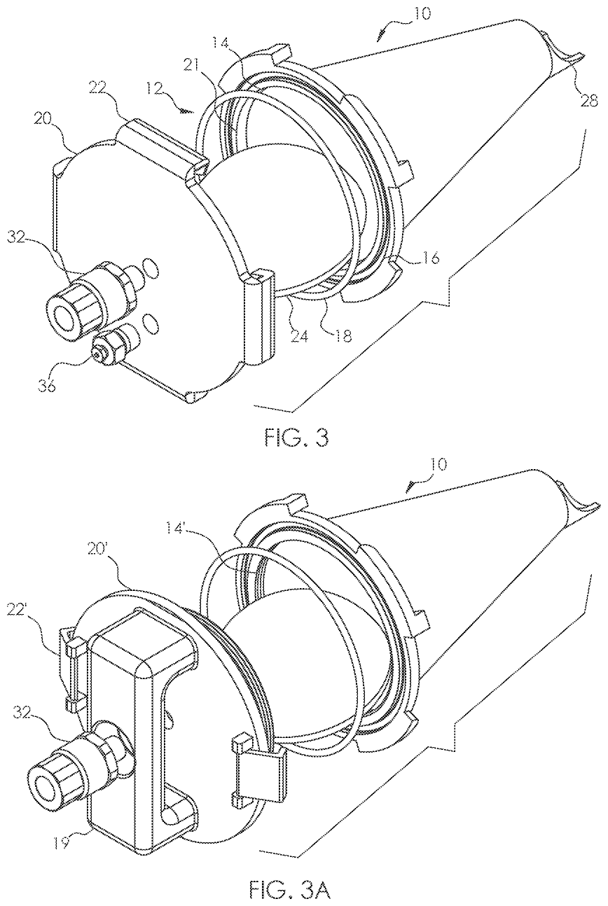

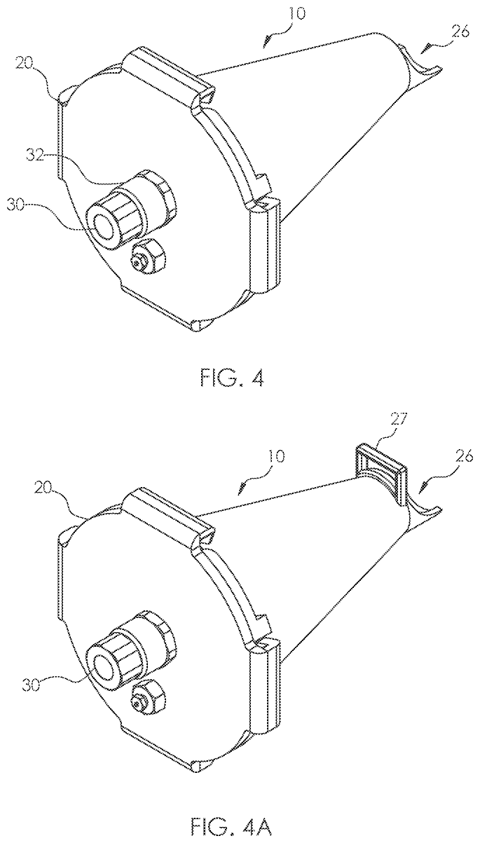

A housing 10 had an inlet 12 with a flange 14 further comprising a groove 16 into which fits an o-ring 18. A cover 20 has spaced axially extending tabs 22 that can be snapped onto flange 14 for a leak tight connection. Other types of quick connections are contemplated such as putting external gaps in tapered flange 14 and advancing tapered tabs 22 through those gaps and then relatively rotating the cover 20 with respect to the housing 10. Furthermore, a radial tapered boss extending from the cover 20 and a mating tapered surface in the respective flange 14 inner diameter can aid with the sealing when the two parts are assembled. This taper 21 can be approximately 8 degrees. Another example is the screw-in-method, shown in FIG. 3A, that entails an internal thread on flange 14′ and external mating threads on cover 20′ that aids in sealing the cover 20′ to the housing 10. A handle 19 on the cover 20′ can aid the operator to rotate and advance the cover 20′ to lock with the sealing surface of the housing flange 14′. Locking latches from the extending tabs 22′ of the cover 20′ can lock onto the housing flange 14′ as another method to seal the cover 20′ to the housing 10. The implant 24 is preferably a breast implant but could be an implant for another location in the body of the patient. The implant 24 is sized to easily slip through inlet 12 directly from the packaging or when manipulated by the surgeon using a gloved hand or a sterile tool. More than a single size of implant 24 can fit into the inlet 12. The outlet 26 can have preferably arcuate taper 28 whose purpose is to open a surgical incision in the patient for access into the surgical pocket in the breast that is also not shown. Taper 28 may also be linear or a combination shape. The opening size of the outlet 26 can vary depending on the incision length in the patient all the while keeping the inlet 12 size within a range of sizes that is allocated for a range of sizes of implants to reduce inventory of covers 20. This outlet 26 can also be a circular, oval, bun shaped or other geometric shapes with or without arcuate taper 28 going radially in or out to accommodate different implant shapes. The outlet 26 can also accommodate a holder 27 for a retractor, shown in FIG. 4A, to rest and lock on top of the delivery device as the retractor supports the skin, soft tissue, and muscle around the surgical site. The retractor can also support the delivery device.

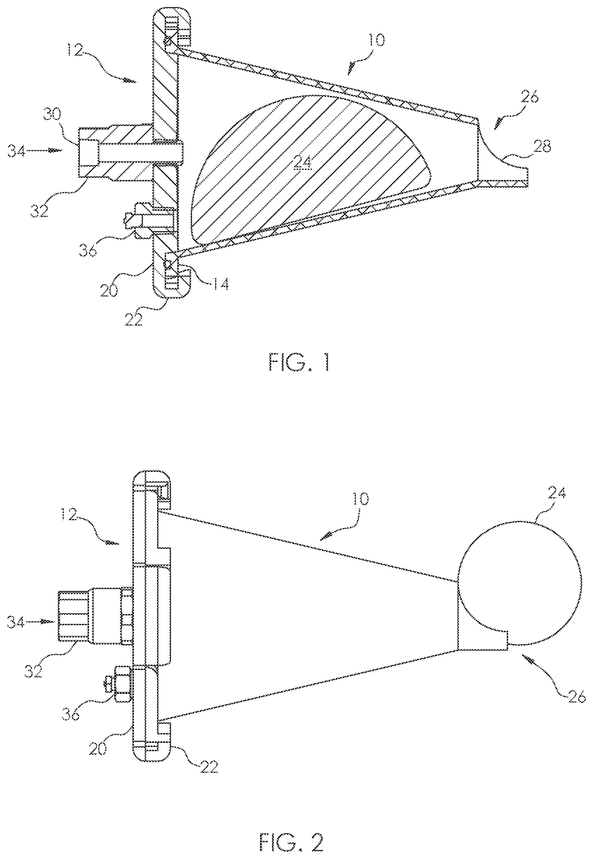

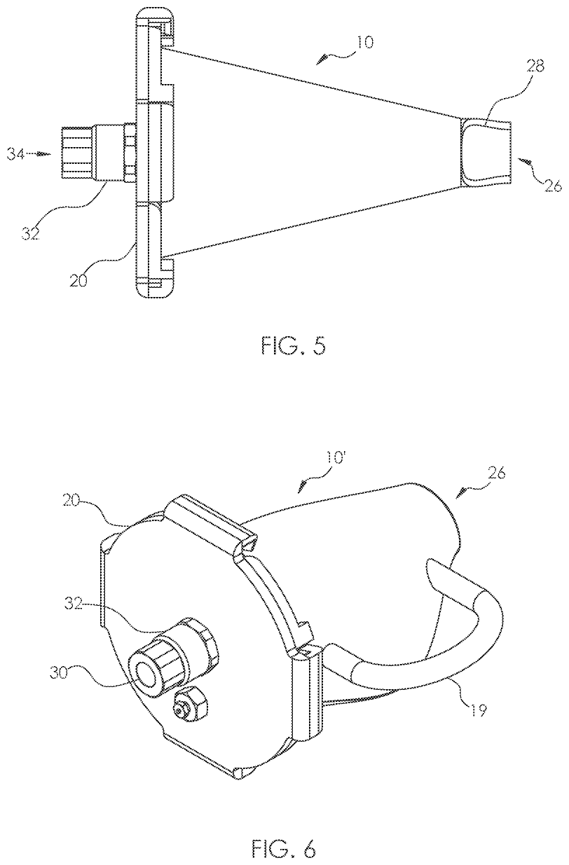

The housing 10 is preferably a transparent conical section as shown in FIGS. 1-5 but may have other shapes such as shown in FIG. 6, for example, to allow the surgeon to observe the implant 24 movement toward the outlet 26. A grip handle 19 can be part of the exterior of the housing 10′ to further make an ergonomic operation for the operator. The low friction interior wall allows for easier implant delivery. The wall material of the housing 10 is sufficiently sturdy to withstand applied pressure, typically within three atmospheres but at times more depending on the application, through connection 30 which features a check valve 32. Cover 20 is also configured to withstand the applied fluid pressure that is expected to be in the range of material characteristic of the delivery device with margin. The walls of the housing 10 while strong enough to contain the applied pressure at connection 30 are still capable of flexing as external pressure is applied but the rigidity prevents advancing the implant 24 by wall squeezing. In essence, the advancement of the implant occurs overwhelmingly by application of a pressurized fluid source represented by arrow 34 connected at connection 30 with mating threads or quick release method. That source could be a compressor, hand operated manual pump, a motor driven pump or a pressurized fluid container, wherein flow from any one of the listed or alternative sources is manipulated by the surgeon or an operating room assistant. The surgeon or the assistant can regulate the fluid delivery and stop such delivery as needed. If the delivery is stopped the check valve 32 holds pressure inside the housing 10 thus keeping the implant in a compressed state but stopping its forward motion toward the outlet 26. The check valve 32 stops the implant 24 from relaxing when pressure from the fluid source 34 is interrupted. This helps the implant 24 from forming folds or creases that can result in a bypassing of the implant 24 by pressurized fluid 34.

As shown in FIG. 6, the wall of housing 10′ is designed to flex when pressurized fluid 34 that can be a liquid, or a gas is applied at connection 30. Alternatively, the shape in FIG. 6 can be an initial shape which is more conforming to the shape of the implant 24 such as with a planar portion to align with a planar back shape of the implant 24 to maintain orientation of the implant 24 on the way out of outlet 26. The anticipated pressure internally is still contained despite some wall flexing at the anticipated internal pressure. A vent valve 36 such as a rupture disc or other type of relief valve on the housing cap creates a fail-safe design in the case the pressure is too high by accident. A manual valve to relieve pressure before releasing cover 20 can also be used.

The material of the housing 10 and the cover 20 is selected from a group that is capable of being autoclaved to temperatures needed to fully sterilize those components for reuse. Reuse is a significant feature of the present invention as it results in considerable cost savings to the surgeon or associated medical practice or hospital as opposed to the above described prior art versions that are of a one and done variety. The housing 10 and cover 20 can be sealingly secured without a sealing element such as o-ring 18 or, alternatively, the sealing element 18 can also be made from a material that can be sterilized in an autoclave. The sealing element can be fitted in a groove as shown or can be flat, so it is secured between the housing 10 and the cover 12. Suitable materials for autoclaving for the housing 10, cover 20 and the seal can be high-temperature thermoplastics, fluoropolymers, stainless steel, titanium. Suitable materials for O-ring 18 can be high temperature silicone. Autoclaving is at a preferred temperature of at least 121-132 degrees Centigrade.

It should be noted that the housing material need not be completely see through or even see through at all. Preferably, if there is a see-through portion it is located nearest the outlet 26.

Those skilled in the art will now appreciate that the present invention allows better delivery control of the implant 24 as compared to the prior designs that required hand force to advance the implant. In the hand squeeze housing design, the implant could reverse movement direction if the grip on the housing was relaxed during delivery. In the present design the outside pressure source eliminated the need to squeeze the housing 10 to allow for a more rigid wall design that can retain the desired pressure. The check valve 32 also allows the implant to hold position without backing up if the delivery pressure is cut off. The fluid pressure delivery system 34 can also include a vent or pressure relief valve to relieve pressure to prevent over pressurization and, if at any time, should disassembly be needed. It is noted that pushing the implant 24 through outlet 26 will also act to relieve any internal pressure in the housing 10. The materials picked for the housing 10, the cover 20 and the seal 18, if used, permit autoclaving after use and allow for reuse in the same or another patient. This is a considerable cost savings to the procedure without putting the patient at any risk from reuse. The described device enables an associated delivery method for implants 24 that better assures implant orientation as the implant 24 moves out the outlet 26 and into the incision. The incision is not shown but is in fact opened wider to receive the implant 24 properly oriented in the surgical pocket after which the incision is closed.

While the above description covers some of the highlights of the apparatus and the associated method, those skilled in the art will appreciate that the full scope of the invention can be determined from the appended claims.

We claim:

1. An implant delivery system, comprising:
at least one implant;
a housing to selectively retain said at least one implant further comprising an outlet through which said at least one implant is delivered;
a fluid pressure source located completely externally of said housing and selectively connected to said housing for delivery of fluid pressure to advance said at least one implant toward said outlet.

2. The system of claim 1, further comprising:
a pressure retention device associated with said fluid pressure source to retain fluid pressure delivered to said housing and against said at least one implant in the event said fluid pressure source is shut off to selectively have said at least one implant hold position in said housing.

3. The system of claim 1, further comprising:
a vent device to deplete pressure in said housing against said at least one implant in the event access to said at least one implant therein is needed.

4. The system of claim 1, further comprising:
a removable cover for said housing further comprising a pressure retention device and a vent device thereon.

5. The system of claim 4, wherein:
said removable cover is quick connected to said housing with at least one of a snap fit or a bayonet mount with relative rotation.

6. The system of claim 1, further comprising:
said housing is at least in part see through near said outlet.

7. The system of claim 1, wherein:
said housing is made of a material that renders said housing reusable after being autoclaved.

8. The system of claim 1, further comprising:
a beveled tip adjacent said outlet that is linear or arcuate or a combination thereof.

9. The system of claim 1, further comprising:
a removable cover connected to said housing with a seal;
at least one of said removable cover and said seal being made of a material that can be autoclaved to make said at least one of said removable cover and said seal reusable.

10. The system of claim 1, wherein:
said at least one implant comprises a plurality of implants;
said housing accommodates said plurality of said implants for sequential delivery.

11. The system of claim 2, wherein:
said pressure retention device comprises a check valve.

12. The system of claim 1, wherein:
said housing comprises a cone shape tapering to said outlet.

13. The system of claim 1, wherein:
said housing has a shape conforming at least in part to the shape of said at least one implant to aid in retaining the orientation of said at least one implant as said at least one implant advances toward said outlet.

14. The system of claim 1, wherein:
said housing is sized to accommodate a plurality of implant sizes through a removable cover, said plurality of implant sizes moving through a given outlet size.

15. The system of claim 1, wherein:
said fluid pressure source delivers up to three atmospheres of fluid pressure and said housing contains said pressure with flexing under elastic deformation.

16. The system of claim 9, wherein:
said housing, cover and seal are made from at least one of high-temperature thermoplastics, fluoropolymers, stainless steel, titanium, and a high temperature silicone.

17. An implant delivery system for a surgeon, comprising:
at least one implant;
a housing to selectively retain said implant further comprising an outlet through which said at least one implant is delivered;
said housing comprising a rigid shape that flexes in elastic deformation when fluid pressure is applied therein and against said at least one implant and said housing is made of a material that renders said housing reusable after being autoclaved.

18. The system of claim 17, wherein:
a fluid pressure source independent of the surgeon selectively connected to said housing for delivery of fluid pressure to advance said at least one implant toward said outlet;
a pressure retention device associated with said fluid pressure source to retain fluid pressure delivered to said housing and against said at least one implant in the event said fluid pressure source is shut off by the surgeon to selectively have said at least one implant hold position in said housing.

19. The system of claim 17, wherein:
said housing comprises a removable cover made of a material that can be autoclaved rendering said removable cover reusable.

20. The system of claim 19, wherein:
said housing comprises a seal made of a material that can be autoclaved rendering said seal reusable.
